(19) United States
(12) Patent Application Publication
(10) Pub. No.: US 2012/0136359 A1
(43) Pub. Date: May 31, 2012

(54) FEMORAL CUTTING GUIDE DEVICE FOR REVISION OPERATIONS IN KNEE ENDOPROSTHETICS

(76) Inventors: Beat Grunder, Worb (CH); Christoph Fankhauser, Solothurn (CH); Walter Supper, Grenchen (CH)

(21) Appl. No.: 13/390,444

(22) PCT Filed: Oct. 28, 2010
(86) PCT No.: PCT/EP10/06610

§ 371 Date: Feb. 14, 2012

(30) Foreign Application Priority Data
Dec. 23, 2009 (DE) 10 2009 060 158.9
Mar. 29, 2010 (DE) 10 2010 013 259.4

(51) Int. Cl. A61B 17/56

(52) U.S. Cl. 606/89

(57) ABSTRACT

A device for the implementation of osteotomies on the distal femur in knee-joint replacement surgery with a base element (11), which comprises an anterior cutting-guide block (12) disposed on the base element (11), which specifies an anterior section plane (14), and a posterior cutting-guide block (13) disposed on the base element (11), which specifies a posterior section plane (16). In this context, the distance between the anterior section plane (14) and the posterior section plane (16) is variable.

The invention relates to a device for the implementation of osteotomies on the distal femur in knee-joint replacement surgery, especially for revision operations.

A total knee prosthesis implanted in the human body has a functional life of approximately 10 to 20 years. An extremely wide variety of causes, especially abrasion of the tibial inlay, loosening of the implant and instabilities of the knee, lead to the failure of the primary total knee prosthesis and require a replacement of the total knee prosthesis in a knee-joint revision operation. In some cases, the removal of the primary total knee prosthesis is associated with massive bone loss, and the resulting marginal conditions for the use of a revision implant are very variable. A wide range of revision instruments is typically required to deal with this diversity of possible defects and events within a revision intervention. As with the implantation of a primary knee-joint replacement, instruments must be provided during the operation, for example, for different femur sizes.

WO 06/042743 A2 describes a ligament-clamping device and a cutting-guide block for preparing the fitting of a joint implant. However, this is not suitable for a revision operation. In this context, a pre-adjustable and re-adjustable resection height are set separately from one another for the medial and the lateral side by means of the ligament-clamping device described, and a corresponding distal osteotomy is implemented using a cutting guide. A further cutting guide, which is selected corresponding to the size of the respective femur, is used, for the implementation of the anterior and posterior section and of the anterior and posterior diagonal section. With typically five femoral implant sizes, five different femoral cutting-guide blocks must therefore be made available. Accordingly, not only different instruments but also different sizes of instruments must be provided for every operation. This makes the operating environment visually cluttered and increases costs through a larger storage requirement.

Accordingly, the object of the present invention is to reduce the number of instruments required in an operation, especially a revision operation, and to allow a simple and intuitive handling of these instruments.

The object is achieved by the device according to the invention as specified in claim 1. The dependent claims specify advantageous further developments of the device according to the invention.

The device according to the invention for the implementation of osteotomies on a distal femur in the context of knee-joint replacement surgery comprises a base element on which an anterior cutting-guide block is arranged which specifies an anterior section plane. Furthermore, a posterior cutting-guide block which specifies a posterior section plane, is arranged on the base element, wherein the distance between the anterior section plane and the posterior section plane is variable. Accordingly, the distance between the anterior and the posterior section plane can be adapted in an advantageous manner to the different femur sizes or respectively femoral-implant sizes. The device according to the invention therefore replaces several conventional femoral cutting-guide blocks and combines these into one. This not only reduces the number of instruments to be prepared for the operation, but also considerably reduces the costs for instruments for the operation.

It is advantageous that a change in the distance between the anterior section plane and a base plane parallel to the latter through the base element is associated with a change in the distance between the posterior section plane and the base plane. In this context, the change in the distance of the anterior section plane relative to the base plane and the change in the distance of the posterior section plane relative to the base plane have a fixed relationship with one another. Accordingly, after the alignment of the base element on the femur, the distances of the anterior section plane and the posterior section plane can be varied together in a single operational step. A separate adjustment of the anterior section plane and respectively the posterior section plane is necessary. The fixed, specified relationship between the changes in the distances of the anterior and respectively posterior section plane relative to one another is adapted to the anatomical size relationships and especially to the dimensions of the different femoral implant sizes. In a further model of the device according to the invention, this relationship can be adjusted appropriately for dimensions of implants from different manufacturers.

The base element advantageously provides a display, which indicates the setting of the distance between the anterior and the posterior section plane corresponding to a size of the femoral implant. This allows the precise and rapid adjustment of the section planes to the selected size of the femoral implant.

The anterior cutting-guide block and the posterior cutting-guide block are advantageously connected by an intermediate element, wherein the intermediate element provides two threads with opposing directions of rotation and different leads. As a result of the thread in the intermediate element, a continuous distance change can be implemented, so that a fine adjustment is possible. Different leads ensure a constant relationship over the entire distance-change range between the displacement of the anterior section plane and the posterior section plane. The relationship of the distance change between anterior and posterior section plane can be varied through the use of different thread leads.

Furthermore, it is advantageous if the base element provides a recess, in which a sleeve can be fixed in a detachable manner. In this context, the recess extends through the base element inclined in the medial-lateral direction by an angle different from zero relative to the perpendicular of a front surface of the base element. A rod-shaped element, which is introduced into the intramedullary channel of the bone, is advantageously held within the sleeve. This allows a fixing of the base element to a characteristic axis of the bone, which subsequently serves for the attachment of the revision implant. With the recess inclined by an angle different from zero, the cutting-guide device according to the invention can be aligned corresponding to the natural Valgus angle relative to the femoral axis. This ensures that the sections in the femur, and subsequently the femoral implant, are aligned perpendicular to the mechanical axis of the femur. This is important, because the weight of the body acts on the knee joint along the mechanical axis.

It is further advantageous that the base element is rotated through an angle β about the perpendicular of the front surface of the base element and fixed. This allows the alignment of the cutting-guide device corresponding to the rotation of the femur, so that the support of the femur on the tibial inlay occurs in a manner corresponding to the original physical conditions. This promotes the stability of the knee-joint replacement as a whole.

A handle, which projects beyond the front surface of the base element in the medial and lateral direction, is advantageously attachable to the base element. With these projecting handles, reference points on the femur, especially the transepicondylar reference points, can be located and covered by turning the handle. Accordingly, the alignment of the base element can be implemented simply and intuitively corresponding to the femur rotation.

A rod-shaped element is preferably introduced and advantageously held in the intramedullary channel of the bone. The rod-shaped element is held eccentrically in the sleeve but parallel to the sleeve axis. This allows a compensation of an offset between the intramedullary channel and the physical axis of the pedestal on the femoral implant for shaft extensions. In this context, the rod-shaped element marks the intramedullary channel. The middle of the sleeve, by contrast, marks the axis of the pedestal of the femoral implant for shaft extensions.

It is advantageous, if the sleeve is rotatable about the axis of the rod-shaped element. On the one hand, this allows an adjustment of the femoral rotation of the cutting-guide device or respectively of the femoral implant and, on the other hand, the implementation through circular translation of an anterior-posterior and medial-lateral fine alignment of the cutting-guide device or respectively of the femoral implant.

A scale is advantageously provided on the anterior cutting-guide block, which displays the medial-lateral width of the femoral implant. By rotating the sleeve within the base element with an offset, that is to say, a sleeve with an eccentrically arranged, rod-shaped element, with a desired position of the anterior femoral section, a medial (for example, 10 o'clock) or a lateral (for example, 2 o'clock) position can be adjusted, so that the marking of the scale agrees as well as possible with the lateral or respectively medial edge of the femur. In this context, the marking is used corresponding to the femoral-implant size already selected in the adjustment of the anterior and posterior section plane.

The anterior cutting-guide block advantageously provides a peg facing in the anterior direction, in which a further cutting-guide block, for example, a distal cutting-guide block, engages. With this plug-on distal cutting-guide block, a so-called cleaning section can be implemented at the distal end of the femur, which is aligned to match the subsequently implemented anterior and posterior sections and/or the femoral implant.

Furthermore, it is advantageous if the anterior cutting-guide block provides a pedestal starting above the anterior section plane, facing in the proximal direction, to which a fixing element is connected. The fixing element in this context is rigidly connected to the femur. Accordingly, additionally to the rod-shaped element, a fixing of the cutting-guide device is guaranteed, especially in the distal-proximal direction.

The device according to the invention therefore allows an optimal alignment of the anterior/posterior cutting-guide block relative to the femur, reduces the number of anterior/posterior cutting guides to only one element for all available femoral implant sizes, allows an intuitive handling for the alignment of the device and forms the mounting and reference for further cutting guides, for example, for the distal bone resection.

Exemplary embodiments of the device according to the invention for osteotomies are illustrated by way of example in the drawings and explained in greater detail with reference to the following description. The drawings are as follows:

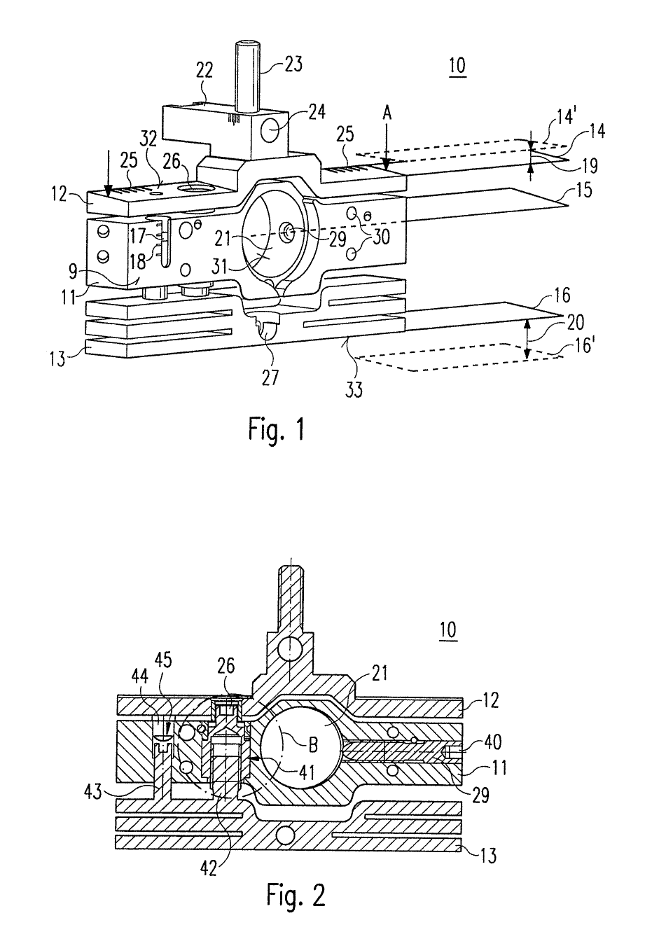

FIG. 1 shows an exemplary embodiment of a cutting-guide device in a perspective view;

FIG. 2 shows an exemplary embodiment of a cutting-guide device according to the invention in a section through the plane A-A;

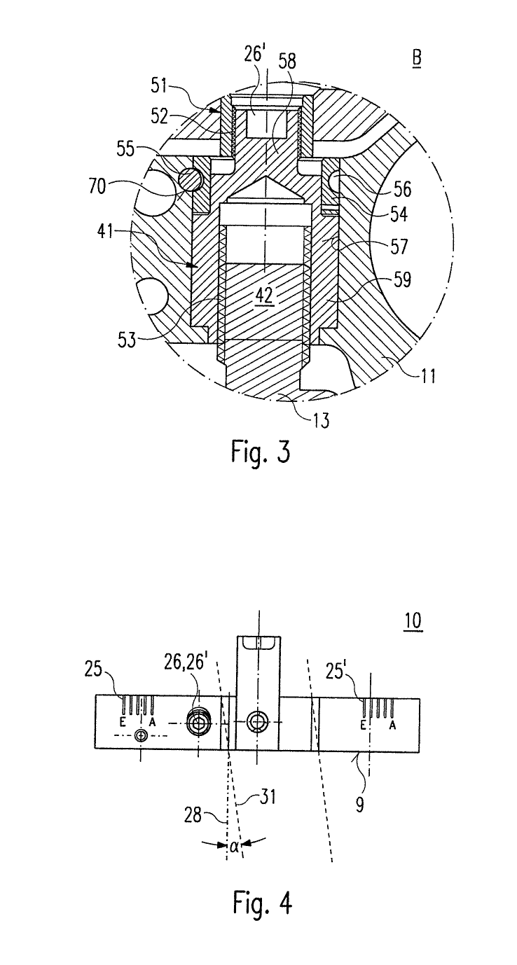

FIG. 3 shows a detail view of an exemplary embodiment according to the invention of an intermediate element;

FIG. 4 shows the exemplary embodiment of a cutting-guide device according to the invention in a plan view;

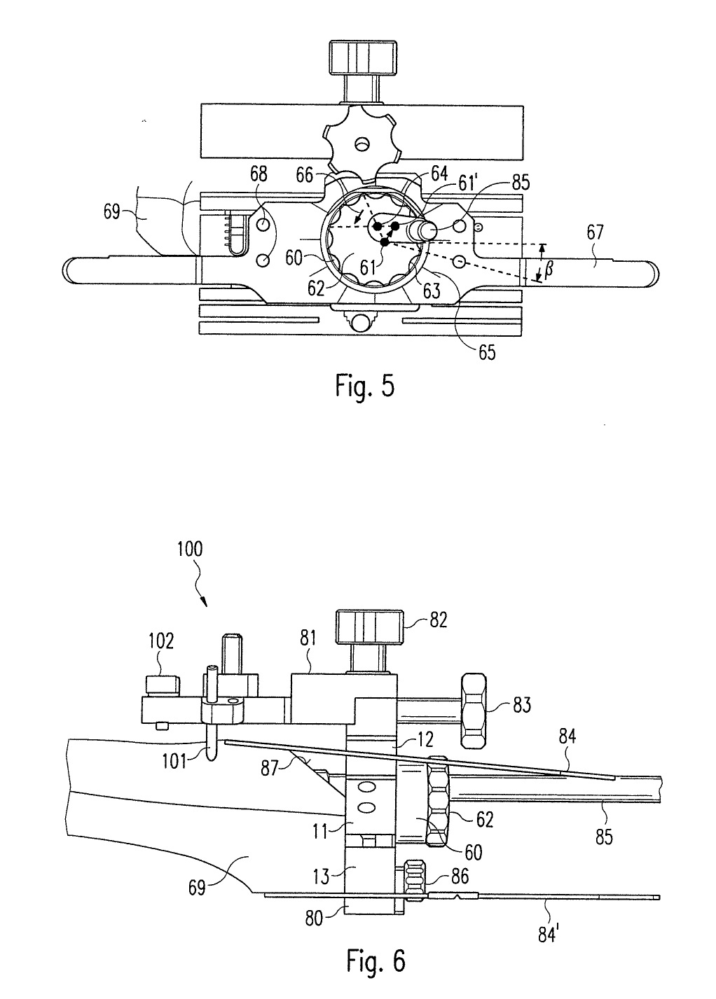

FIG. 5 shows the exemplary embodiment of a cutting-guide device according to the invention fitted to a bone with the viewing direction towards the front end of the cutting-guide device;

FIG. 6 shows a lateral view of the exemplary embodiment of a cutting-guide device according to the invention fitted to a bone;

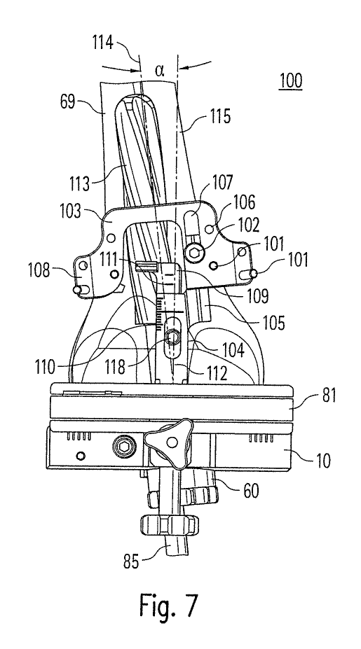

FIG. 7 shows a plan view of the exemplary embodiment of a cutting-guide device according to the invention fitted to a bone.

Parts corresponding to one another have been indicated with the same reference numbers in all the drawings.

The sides and direction markings are presented in the following description with reference to the specified fitting position of the cutting-guide device on the bone and the position of the bone in the body. The device according to the invention is disposed with its rear side disposed opposite to the front side at the distal end of the femur, perpendicular to the mechanical axis between the knee and the hip joint on the femur. All other position markings relate to this basic position.

FIG. 1 shows an exemplary embodiment of a cutting-guide device 10 according to the invention in a perspective view with the viewing direction towards the front side of the cutting-guide device. The cutting-guide device comprises a base element 11, on which an anterior cutting-guide block 12 aligned towards anterior and a posterior cutting-guide block 13 aligned towards posterior are arranged. The base element 11 provides a first recess 21, of which the internal surface 31 passes through the base element 11 inclined in the medial-lateral direction by an angle not equal to zero from the perpendicular to the front surface. Dependent upon this inclined first recess 21, different cutting-guide devices are provided for the left and respectively right knee, wherein the first recess 21 is directed towards lateral in each case. Otherwise, the alignment of the cutting-guide device 10 remains the same.

A sleeve 60, which is illustrated in FIGS. 5, 6 and 7, can be inserted into the first recess 21. The sleeve 60 is fixed in its position by a holding pin 29, which can be rotated into the first recess 21. The base element 11 also provides boreholes 30 at the front side, into which additional components of the cutting-guide device 10 can be fixed.

A posterior cutting-guide block 13 is connected to the base element 11. A third recess 27 in the posterior cutting-guide block 13 serves to receive a posterior cutting guide, as illustrated in FIG. 6. On the side disposed opposite to the posterior cutting-guide block, an anterior cutting-guide block 12 is attached to the base element 11. On the side of the anterior cutting-guide block 12 facing in the anterior direction, a scale is provided, which indicates the medial-lateral size of the femoral implant. A corresponding scale is provided on the medial and also the lateral side of the anterior cutting-guide block 12. Approximately in the middle of the anterior cutting-guide block 12, a pedestal 22 is formed, which extends in the anterior direction and projects in the proximal direction beyond the anterior cutting-guide block. The pedestal 22 serves as a support and attachment element for further cutting-guide elements and for the attachment of a fixing element, as illustrated by way of example in FIG. 7. A second recess 24 penetrates the pedestal 22 and allows the introduction of a connecting element 83, see FIG. 6. A peg 23, which points in the anterior direction, projects above the pedestal 22. Further cutting-guide elements, such as the cutting-guide element 81 in FIG. 6, are fitted and fixed to the peg 23.

The upper side 32 of the anterior cutting-guide block defines the anterior section plane 14. In a similar manner, the lower side 33 of the posterior cutting-guide block 13 defines the section plane 16. For the implementation of the osteotomy, a saw blade is placed respectively against this section plane 14, 16 and guided along the section plane 14, 16. In order to adapt the cutting-guide device 10 according to the invention for different femur sizes, the anterior section plane 14 and the posterior section plane 16 are displaceable.

In the illustrated exemplary embodiment, the position of the two planes 14 and 16 relative to the parallel base plane 15, which passes through the middle of the first recess 21, is displaced by rotating an adjustment tool, which is introduced into the recess 26. The distance 19 between the original anterior section plane and the newly adjusted anterior section plane 14′ and the distance change 20 between the original posterior section plane 16 and the newly adjusted posterior section plane 16′ have a fixed relationship to one another. A typical relationship between the anterior distance change and the posterior distance change is disposed around 2:5. Through a display on the front side 9 of the base element 11, which indicates the femoral implant sizes, and a corresponding adjustment marking 17, the anterior section plane and respectively the posterior section plane can be adjusted to the distances corresponding to the size of the femoral implant. Accordingly, no further cutting-guide elements are required for the different femoral-implant sizes. For example, with five different femoral-implant sizes, four operating instruments fewer need to be provided.

FIG. 2 shows a section through the plane of the exemplary embodiment illustrated in FIG. 1, which extends through the points A-A in the direction of the arrow. The holding pin 29, which is accessible from the lateral surface of the base element 11 with an adjustment aperture 40, and extends up to the first recess 21, is disposed in the base element 11. On the side of the base element 11 disposed opposite to the holding pin 29, a posterior guide element 43 and an anterior guide element 44 engage with the base element 11. These guide elements are used for the stabilisation of the anterior and respectively posterior cutting-guide blocks 12, 13. The posterior guide element 43 provides an end stop 45 with an annular marking at its anterior end. This is visible at the front side 9 of the base element 11 and forms the adjustment marking 17 for the display 18 of the femoral implant size.

The anterior cutting-guide block 12 and the posterior cutting-guide block 13 are coupled by an intermediate element 41. The intermediate element is mounted in a rotatable manner in the base element 11 but is fixed in the axial direction.

The detail marked with letter B is shown in enlargement in FIG. 3. The intermediate element 41 is introduced into a recess 57 of the base element 11 and is enclosed by a split washer 54. The split washer 54 provides a recess 56 in the periphery, in which a holding pin 55 engages. The holding pin 55 in this context is pushed into a recess 70, which penetrates the base element 11 in the distal-proximal direction and is pushed into the recess 56 in the intermediate element 41. Accordingly, the intermediate element 41 is fixed in the axial and respectively anterior-posterior direction.

A head region 58 of the intermediate element 41 is provided with a first peripheral thread 52. This engages in a thread, which is connected to the anterior cutting-guide block 12. This thread can be cut either into the anterior-cutting-guide block 12 or can engage through a threaded sleeve 51, which is introduced in a borehole in the anterior cutting-guide block 12 in a force-fit or form-fit manner. The foot region 59 of the intermediate element 41 contains a recess, which provides a second thread 53. A threaded peg 42 of the posterior cutting-guide block engages in the second thread 53. The first thread 52 and the second thread 53 provide opposing directions of rotation, that is to say, they are designed as clockwise and respectively anticlockwise threads. Similarly, the lead of the first thread 52 and of the second thread 53 is different, so that the propulsion on the anterior side provides the desired relationship relative to the propulsion on the posterior side.

FIG. 4 shows a plan view of the exemplary embodiment of a cutting-guide device 10, as illustrated in FIGS. 1 and 2. Accordingly, the display of the medial-lateral femoral implant size 25, 25′ is clearly visible. With reference to this display, the cutting-guide device 10 is brought into agreement with the lateral and respectively medial edge of the femur. This monitors and ensures that the femoral implant is positioned appropriately on the femur in the medial-lateral direction for the femur size. Similarly, the recess 26 for the insertion of an adjustment tool 26 is visible. The intermediate element 41 is readily accessible and easily adjustable through this recess.

This illustration shows the inclination of the first recess 21 in the medial-lateral direction relative to the perpendicular 28 to the front surface 9. The internal surface 31 of the first recess 21 is shown extended by the dotted lines. This is inclined by the angle α relative to the perpendicular 28 to the front surface 9. The angle α in this context corresponds to the Valgus angle by which the femur is inclined relative to the physical axis 115. Accordingly, with a fixing of the cutting-guide device 10 in the intramedullary channel 114, it can be ensured that the osteotomies, especially the distal osteotomy, is arranged perpendicular to the physical axis 115. The position of the named axes is presented in FIG. 7.

FIG. 5 shows the exemplary embodiment of a cutting-guide device 10, fitted to the distal femur in the viewing direction towards the front side 9 of the cutting-guide device 10. A handle 67 is attached to the base element 11 and projects in the medial-lateral direction beyond the base element 11. The cutting-guide device 10 is aligned corresponding to the femoral rotation by rotating the handle 67 and aligning with prominent bone formations of the distal femur. For the attachment of the handle 67 to the base element 11, the pins 68 in the handle 67 are inserted into the boreholes 30 in the base element 11, see FIG. 1. A rotation through the angle β in the clockwise direction, which corresponds to the femoral rotation about the centre 61 of the sleeve 60, starting from the illustrated starting position of the handle 67, is shown in FIG. 5.

In order to compensate an offset of the starting position of the physical axis 115 from the penetration point of the intramedullary channel towards the distal end of the femur, the rod-shaped element 85 is guided through a recess formed in an eccentric manner in the sleeve 60. The distance between the centre of the sleeve 61 and the centre 64 of the rod-shaped element 85 corresponds to the offset of the physical axis 115 relative to the intramedullary channel 114. For the fine tuning of the position of the femoral implant in the anterior-posterior or medial-lateral alignment, the sleeve is mounted in a rotatable manner about the rod-shaped element 85. Markings 63 on the head 62 of the sleeve and markings 65 on the handle 67 are arranged distributed around the periphery of the sleeve 60 and facilitate the reproduction of the anterior-posterior or medial-lateral position for subsequent fitting of the femoral implant. FIG. 5 shows a rotation 66 of the sleeve 60 about the centre 64 of the rod-shaped element. This rotation 66 brings about a relocation of the centre 61 of the sleeve 60 to the position 61′ and accordingly a displacement of the cutting-guide device 10 in the illustrated direction and by the illustrated distance.

After the adjustment of the femur size, the femoral rotation and the offset between the physical axis 115 and the intramedullary channel 114 at the distal end of the femur, the cutting-guide device 10 is fixed via a fixing device 100, which is hooked into the pedestal 22 or respectively the peg 23. Reference is made to FIG. 7 here. Similarly, a further cutting-guide block 81 is attached for the implementation of the distal bone resection and fixed via a second 83 and third 82 connecting element to the cutting-guide device 10. The fixing device, and accordingly the distal position of the cutting-guide device 10, is fixed via anchor-holding elements 101.

In a revision operation, a distal cleaning section is conventionally implemented, as the first stage, with the assistance of the distal cutting-guide block 81. Following this, the cutting-guide device 10 is brought flush to the distal section. For this purpose, a fourth connecting element 102 on the fixing device 100 is released. For the implementation of the posterior [??] resection, a saw blade 84 is guided, as illustrated, along the anterior section plane of the anterior cutting-guide block 12. The anterior section plane is conventionally inclined upwards in order to remove as little bone material as possible. In order to guide the posterior section, a posterior cutting guide 80 can optionally be attached by means of a first connecting element 86 on the posterior cutting-guide block 13. This prevents the saw blade 84′ from slipping.

In order to implement a distal diagonal section 87, the cutting-guide device 10 is released from the fixing device 100 and removed together with the rod-shaped element 85 from the femur 69. A cutting-guide block for the anterior diagonal section 87 is held only by the fixing device 100.

The fixing device 100 is illustrated in greater detail in FIG. 7. An anchor 103 extending over the medial-lateral extension of the femur is aligned perpendicular to the intramedullary channel 114, which is indicated approximately with the axis of the reamer 113. Anchor holding elements 101 are connected to the femur 69 via boreholes 106. As an alternative, the anchor 103 can be orientated and fixed via the cutting-guide device 10, which is aligned in the intramedullary channel 114 via the rod-shaped element 85 or respectively the reamer 113 attached to the latter. A displaceable connection between the anchor 103 and the guide rail 105 is provided via a fourth connecting element 102, which engages through a groove 107 in the anchor 103 and is connected to a guide rail 105. For example, a second cutting-guide element 81, which is aligned perpendicular to the coupling shaft 112, is attached via a coupling shaft 112.

In order to allow a displacement of the coupling shaft 112 towards distal or proximal, relative to the anchor 103 with a fixed cutting-guide device 10, the coupling shaft 112 and the guide rail 105 in the exemplary embodiment are inclined by an angle different from zero, which corresponds to the Varus angle α.

For the further attachment of the anchor 103 to the femur 69, the lateral regions 108 of the anchor 103 disposed externally in the medial-lateral direction are inclined towards posterior. The boreholes 106 in the inclined lateral region 108 accommodate additional anchor-holding elements 101, which serve as a depth stop for the saw blade with given osteotomies. The probe rail 109 can optionally be introduced into the coupling shaft 112 and is used for probing the anterior bone and accordingly for the correct anterior-posterior positioning of the cutting-guide device. If the probe rail 109 touches the femur anteriorly, the anterior section with the inclined saw blade 84, and accordingly the anterior termination of the femoral component will come to be disposed at the correct height. The selected size of the femoral implant is adjusted with the scale 111. The probe rail 109 must be adjusted on the scale 111 to the marking corresponding to the femoral-implant size.

All of the features described and/or illustrated can be advantageously combined with one another within the framework of the invention. The invention is not restricted to the exemplary embodiment described.

1.-20. (canceled)

21. A device for the implementation of osteotomies on the distal femur in knee-joint replacement surgery, comprising:
a base element;
an anterior cutting-guide block disposed on the base element, the anterior cutting-guide block defining an anterior section plane; and
a posterior cutting-guide block disposed on the base element, the posterior cutting-guide block defining a posterior section plane;
wherein the distance between the anterior section plane and the posterior section plane can be altered.

22. The device of claim 21, further comprising:
a first variation of the distance between the anterior section plane and a base plane through the base element; and
a second variation of the distance between the posterior section plane and the base plane;
wherein the first variation and the second variation have a fixed relationship with each another.

23. The device of claim 21, wherein the base element provides a display which displays the adjustment of the distance between the anterior section plane and the posterior section plane corresponding to the size of a femoral implant.

24. The device of claim 21, further comprising a scale which displays the medial-lateral width of a femoral implant, wherein the scale is fitted on the anterior cutting-guide block.

25. The device of claim 21, wherein the anterior cutting-guide block and the posterior cutting-guide block are connected by an intermediate element having two threads with opposing rotational directions and different leads.

26. The device of claim 25, wherein the intermediate element is configured to be mounted in a rotatable manner in the base element and fixed in the axial direction.

27. The device of claim 25, wherein the intermediate element contains a recess for receiving an adjustment tool.

28. The device of claim 21, wherein a posterior cutting guide can be removably attached to the posterior cutting-guide block.

29. The device of claim 21, wherein the base element contains a sleeve recess configured to receive a removably attachable sleeve.

30. The device of claim 29, wherein the sleeve recess extends through the base element and is inclined in the medial-lateral direction by an angle different from zero relative to a plane that is perpendicular to a front surface of the base element.

31. The device of claim 29, wherein a rod-shaped element is held in the sleeve.

32. The device of claim 31, wherein the rod-shaped element is introduced into and held within the intramedullary channel of a bone of a patient.

33. The device of claim 29, wherein the base element is fixed to the sleeve and rotated about an axis through the center of the sleeve by an angle different from zero.

34. The device of claim 21, wherein a handle can be attached to the base element and projects in the medial and lateral direction beyond a front surface of the base element.

35. The device of claim 29, wherein the sleeve is fixed on the base element by a holding pin configured to be introduced into the sleeve recess.

36. The device of claim 35, wherein the rod-shaped element is held in the sleeve in an eccentric manner but parallel to the sleeve axis.

37. The device of claim 36, wherein the sleeve is rotatable about the axis of the rod-shaped element.

38. The device of claim 21, wherein the anterior cutting-guide block defines a pedestal starting above the anterior section plane facing in the proximal direction to which a fixing element is connected.

39. The device of claim 38, wherein the fixing element is connected rigidly to the femur.

40. The device of claim 21, wherein the anterior cutting-guide block contains a peg facing in the anterior direction into which another cutting-guide block engages.
